(19) United States
(12) Patent Application Publication
(10) Pub. No.: US 2017/0364868 A1
(43) Pub. Date: Dec. 21, 2017

(54) SYSTEM OF ATTENDANCE AND TIME TRACKING WITH REPORTING

(71) Applicant: THUMBTAG INDIA PRIVATE LIMITED, Mylapore Chennai (IN)

(72) Inventor: Srikanth Ramachandran, Tustin, CA (US)

(21) Appl. No.: 15/186,291

(22) Filed: Jun. 17, 2016

(51) Int. Cl. G06Q 10/10; A61K 8/34; A61Q 19/10; G06K 9/00

(52) U.S. Cl. CPC G06Q 10/1091; G06K 9/00006; A61K 8/34; A61Q 19/10

(57) ABSTRACT

A system for biometric staff management for a physical jobsite is disclosed. It uses a centralized reporting server and a multi-purpose computing device having authentication software and in communication with at least one biometric sensor. The jobsite workers who are users of said software authenticate into the system by using the at least one biometric sensor.

### BACKGROUND OF THE INVENTION

### 1. Field of the Invention

The field of the invention is a system for managing attendance and presence of individuals on a work site without relying on physical objects such as smart cards, RFID cards, swipe cards, and others.

### 2. Background of the Invention

In various embodiments, the invention provides a device and system for accounting for personnel at a job site, along with a stand, which allows for accurate accounting of employees, is resistant to fraud, and provides a stable physical stand to interact with the system.

In one embodiment, the invention comprises a central server, at least one remote employee tracking device, an authentication device, and at least one stand for the devices. The server provides the logic, while employee tracking device provides a physical location for employees to interact with the system. The system further comprises a moveable, in one embodiment, stand which is rigid and level, which allows for the employees to interact with the system in a time efficient manner. The authentication device prevents fraud by relying on biometric recognition of employees, in one embodiment. In other embodiments, the authentication device also uses photo verification, in conjunction with or separate from voice authentication.

Traditionally, employee time has been accounted for using paper records. Beyond paper records, computerized employee tracking systems generally rely on a physical object, such as a key card or smart card. The systems require the employee to authenticate into the system and while the systems are resistant to fraud, they can still be cheated. Other embodiments rely on employees' phones and specialized apps to allow employees to clock in and out. However, that approach requires each employee to have a functioning and charged phone and still is subject to fraud. In many jobsites there are large numbers of temporary workers and supervisors may not know who will come during any one shift. Further, the workers will move from one job site to another, as needed. Assigning ID tags or other physical tokens would not only be difficult and prone to fraud, the overhead created by physical item authentication would create more overhead than benefit.

A need exists in the art for a system that allows for accounting for employee location at a job site without relying on physical objects assigned to each employee and one that is highly resistant to fraud.

### SUMMARY OF INVENTION

An object of the invention is to create a system for accounting for employees. An advantage of the invention is that it allows for an instantaneous determination of which employees are on a job site. An advantage of the invention is that it enables highly accurate tracking of employee presence.

Another object of the invention is to allow for highly accurate tracking of employees. A feature of the invention is real-time presence tracking. An advantage of the invention is that it enables notification of employees for purposes of task management, in the event of a problem at the jobsite.

An additional object of the invention is to account for employees in the event of a job site event or disaster. A feature of the invention is that it allows for supervisors to know the location of employees in real time and the number of employees at any one job site. An advantage of the invention is that it allows for appropriate responses to adverse events.

A further object of the invention is to provide a system to track employees without relying on physical devices or employee input. A feature of the invention is that it uses biometric information to authenticate users without relying on physical objects, nor does it require users to enter passwords. An advantage of the invention is that it users can authenticate into the system quickly and with minimal interaction with the system obviating the need for reading and writing.

An object of the invention is to provide a means to provide a highly stable physical location for the employees to interact with the system. A feature of the invention is that it includes a stand which can be located anywhere on a job site and the stand is highly stable. An advantage of the invention is that the employees can authenticate into the system in a convenient location in a time-efficient manner.

Yet another object of the invention is to provide multiple locations for employees to interact with the attendance system. A feature of the invention is that it uses inexpensive components which can be deployed in multiple locations. An advantage of the system is that employees do not have to waste time waiting to use a single system to clock in and out.

A further object of the invention is to provide a system which can be deployed on demand. A feature of the invention is that the authentication stations can be deployed in multiple locations and can be moved as demand for the authentication stations changes. A benefit of the system is that it allows management to deploy an optimal number of authentication points.

Another object of the invention is to provide a system for generating reports on employees. A feature of the invention is that the centralized server can generate and display a number of reports regarding employee attendance and other employee-related tasks. A benefit of the system is that real-time or nearly real-time reporting regarding employees is available to management.

An additional object of the invention is to provide a point for employees to clean their hands before interacting with the biometric sensor. A feature of the system is that the stand includes a hand cleaning dispenser. A benefit of the system is that employees are reminded to clean hands at the beginning of their work shift and the use of an optimum amount of hand cleaning solution improves the functioning of the biometric sensor.

A further object of the invention is to provide a system for centralized searching of employees on a job site. A feature of the system is that in one embodiment the centralized server provides for a means to search for employees physically present on the job site. A benefit of the system is that it allows for efficient location of employees and resources on a job site.

An additional object of the invention is to provide a system which uses a multi-purpose computing device. A feature of the invention is that the attendance tracking software runs as an application on a standard phone or tablet. A benefit of the invention is that in some embodiments, the system allows for use of standard computing equipment.

A further object of the invention is to provide a system which sends alerts regarding attendance issues. A feature of the system is that the centralized reporting server automatically sends alerts if certain threshold values are crossed, such as the number of absent employees, or absence of a certain class of employees (such as managers, forepersons, or manual laborers). A benefit of the system is that it can ensure that both the required employees and staff levels are maintained at a job site and further prevents fraud by swiping in and out at unauthorized locations.

An additional object of the invention is to notify end users where an end user should report. A feature of the system is that upon swiping into a system, an end user is prompted to proceed to their assignment for the day. A benefit of the system is that it allows for staffing of tasks on a job site based on real-time presence, preventing the unnecessary transit of employees between remote tasks on a job site.

A further object of the invention is to provide a system to de-authenticate users. A feature of the system is that users are prevented from checking in to a location if the end users attempt to authenticate to a location without permission to do so. A benefit of the system is that management can, in real time, notify end users if they are authenticated on a job site.

An additional object of the invention is to provide a system for automated accounting of the cost of a project. A feature of the system is that the workplace costs associated with a project may be calculated in real time. A benefit of the system is that cost projections and actual real-time costs can be compared on demand.

An additional object of the invention is to ensure that sub-contractors are provisioning the type and quantity of employees promised in a contract. A feature of one embodiment of the system is that contractors are held accountable for the number of workers as well as the qualifications of workers present on a job site. A benefit of the system is that sub-contractors are held accountable.

A system for managing attendance at a job site is described. The system comprises a centralized reporting server; a multi-purpose computing device having authentication software and in communication with at least one biometric sensor; wherein jobsite workers who are users of said software authenticate into the system by using the at least one biometric sensor; wherein said software provides live status of workers present on the physical jobsite; wherein said software provides daily swipe in and swipe out information for each worker; wherein said software transmits status and swipe in information to said centralized reporting server and exchanges other information with the reporting server; and a stand for said multi-purpose computing device and at least one biometric sensor; wherein said stand further comprises a hand cleaning solution dispenser; wherein said stand provides a rigid base for the multi-purpose computing device and at least one biometric sensor; wherein said centralized reporting server provides information on the workers present on the jobsite, a total cost for workers, and contractor analysis.

### BRIEF DESCRIPTION OF DRAWING

The invention together with the above and other objects and advantages will be best understood from the following detailed description of the preferred embodiment of the invention shown in the accompanying drawings, wherein:

FIG. 1 depicts an overview of the system pursuant to one embodiment of the invention;

FIG. 2 depicts an overview of an embodiment of the invention;

FIGS. 3A-D depicts an overview of an interface of the invention;

FIGS. 4A-C depict another view of an embodiment of an interface of the invention;

FIGS. 5A-B depicts another view of an embodiment of an interface of the invention;

FIGS. 6A-F depict views of several functions of an embodiment of the invention; and

FIGS. 7A-D depict a view of one embodiment of the data store of the invention.

### DETAILED DESCRIPTION OF THE INVENTION

The foregoing summary, as well as the following detailed description of certain embodiments of the present invention, will be better understood when read in conjunction with the appended drawings.

To the extent that the figures illustrate diagrams of the functional blocks of various embodiments, the functional blocks are not necessarily indicative of the division between hardware circuitry. Thus, for example, one or more of the functional blocks (e.g. processors or memories) may be implemented in a single piece of hardware (e.g. a general purpose signal processor or a block of random access memory, hard disk or the like). Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, an element or step recited in the singular and proceeded with the word “a” or “an” should be understood as not excluding plural said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

### System Overview

Briefly, while the system is described as a system and method of tracking employees on a job site, the same system can be deployed in a non-work environment. For example, the system may be used to track students on a school campus. Other embodiments can be used to track residents of institutions, guests at a hotel, and the like. The system can also be used to control access to locations, such as requiring swiping into the system to open a door locked with an electronic lock.

For purposes of conciseness, the system will be described as tracking attendance of employees on a job site, such as an outdoor construction site.

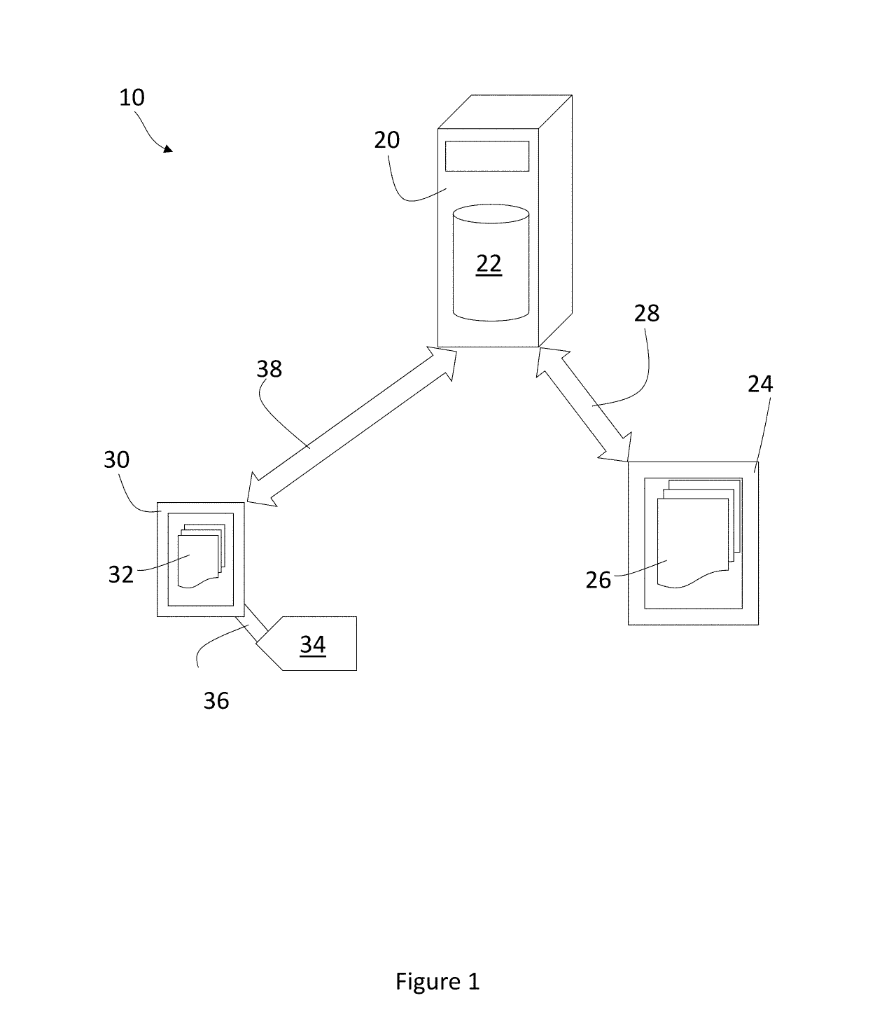

Turning to the figures, FIG. 1, shown therein is a schematic overview of one embodiment of the system. The system 10 comprises a centralized server 20 having a data store 22. In one embodiment, the centralized server 20 is a single on-site server, in another embodiment, the centralized server 20 comprises shared time on a multi-user service such as in a cloud computing service. The data store 22 comprises a suitable persistent data store, such as a relational database or other appropriate data store. In one embodiment, this is implemented as a single database made available to the client devices by a database management system.

As shown in FIG. 1, the system 10 includes a server interface 26 which runs on a system 24 which operates using secure communication channel 28 with the server 20. In one embodiment, the system 24 comprises a multi-purpose computer and the server interface 26 comprises a secure website.

The system 10 also includes an end user device 30 having an end user interface 32. The interface 32 displays information for the user as well as providing instructions on how to interact with the biometric reader 34 in direct communication 36 with the user device 30. The end user device 30 includes a communications interface 38 to the server 20. In one embodiment, the end user device 30 is a portable phone with a touchscreen and the communications interface 38 comprises the phone's internet connection.

While in FIG. 1, the end user device 30 and the server 20 are shown as communicating directly using the link 38, in some embodiments the communications between the end user device 30 and the server is mediated over a multi-user network, such as the Internet.

Further, while in FIG. 1 only one end user device 30 and coupled biometric sensor 34 is shown, in other embodiments, several end-user devices 30 are communicating with the server 20 at a single job site.

### End User Device

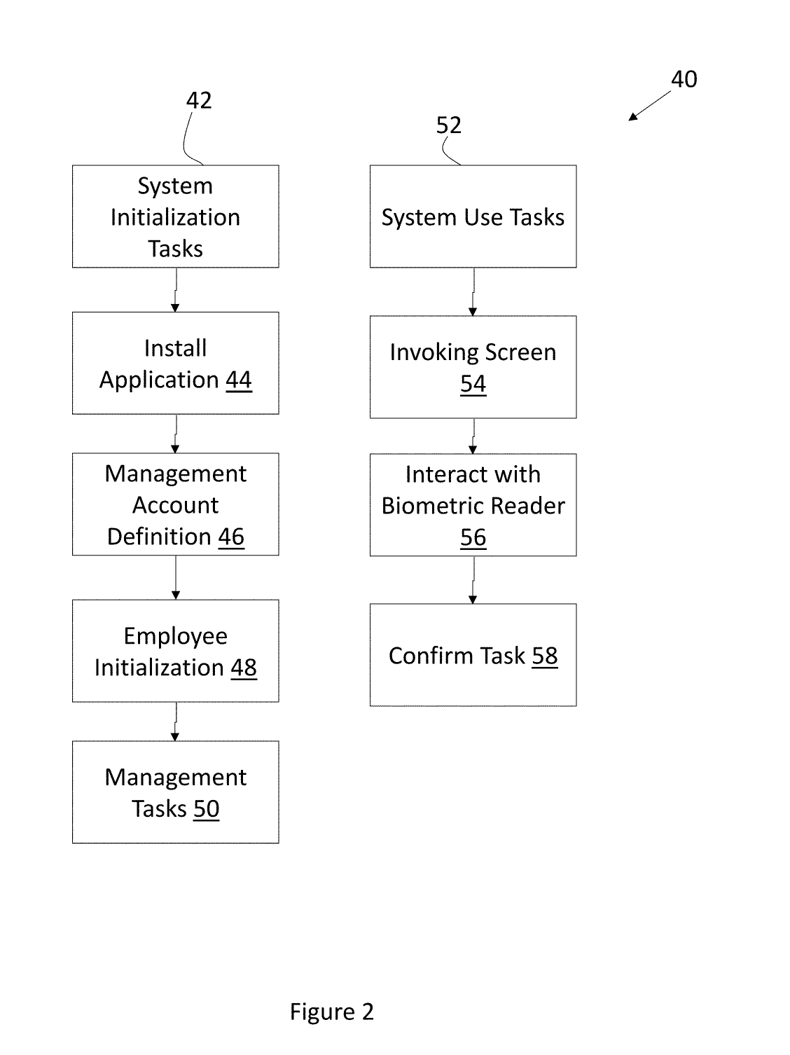

FIG. 2 depicts a flow chart depicting the operation of an application pursuant to one embodiment of the invention, running on the end user device 30. The application pursuant to one embodiment comprises two modes of operation. Inasmuch as the system and application are designed to operate with a standard device, such as a tablet or smartphone, the device must be initialized for use with the system. While the device requires initialization, the device does not need to be totally devoted to use in conjunction with the application, and the application is one of several programs running on the end user device, in one embodiment.

In one embodiment, the end user device 30 include a geographic location system, which uses a combination of geo-location technologies to ensure that the end user device 30 is found at a job site. The end user device 30 includes a receiver for location services such as GPS, GLONASS, and Galileo systems. The end user device 30 also includes an accelerometer to determine whether it is being moved at an unscheduled time. Further, the system 10 includes a record of immutable identifiers, such as IMEI numbers for phone devices to ensure that unauthorized devices do not provide data to the data store. By storing IMEI identifiers for each phone, the system can ensure that it hasn't been substituted and by recording the geolocation of each phone the system ensures that the phones have not moved.

The system initialization tasks 42 begin with the installation 44 of the end user application 40. Following installation, a management account is created and one or more management users are defined 46 on the end user device 32. It should be noted, that in one embodiment, the end users do not need to be set up accounts on the device; only a management account is created and requires authentication, such as by use of a username and password or by use of biometric information.

Upon creation of the management account 46, the management account user can define and configure employees 48 who will use the end user device 30. In one embodiment, the employee initialization allows for bulk registration of employees who will use the end user device 30. In one embodiment, the bulk registration is performed by importing in the system data from other legacy payroll systems, such as by using XML, a text file, or other means of data exchange.

The bulk registration allows for registration of multiple workers. When the system is first installed on a job site, biometric information is collected from all workers and entered into the system. The biometric information is entered using the client biometric device 34 in one embodiment. In another embodiment, the secondary interface 26 is used to perform bulk registration of workers.

A benefit of the system is that the client device 32 and biometric device 34 can detect new employees and allow for instant registration, including automated creation of their biometric record and their photographic and voice print record. The system 10 ensures that only authorized users can allow new workers to register. The system ensures that job sites with high employee turnover, and with employees with similar names, will nonetheless accurately reflect who has come to work and who is present on the job site.

Management tasks 50 also include the ability to edit existing workers, to swipe workers in and out, and to force the workers out. Forcing the workers out is performed at the end of the business day, in one embodiment. At the end of the designated interval, such as a shift or work day, if the operator manager determines that certain workers are no longer present on the job site but nonetheless failed to swipe out before leaving, the operator can use the forced swipe out the errant employees. Further, the system will log this information so that upon returning to the job site the employees are reminded of the need to properly swipe in and out. The system also allows to synchronize the data on demand. Synchronization is necessary because the end user device 30 may not have a constant connection to the central server 20 shown in FIG. 1.

A further management task 50 is to change the account password and other account tasks, including closing down the account.

Even on the end user device 30, when in the management mode, the end user device 30 can generate some reports, such as the workers that are on site, as part of the management tasks 50, in one embodiment. More reporting capabilities are part of the management interface, described below.

The use of the system tasks 52 comprise invoking the swipe in and out screen 54. The end user thereafter interacts with the biometric reader following a prompt 56 on the screen. Thereafter the end user accepts the swipe in or swipe out confirmation 58.

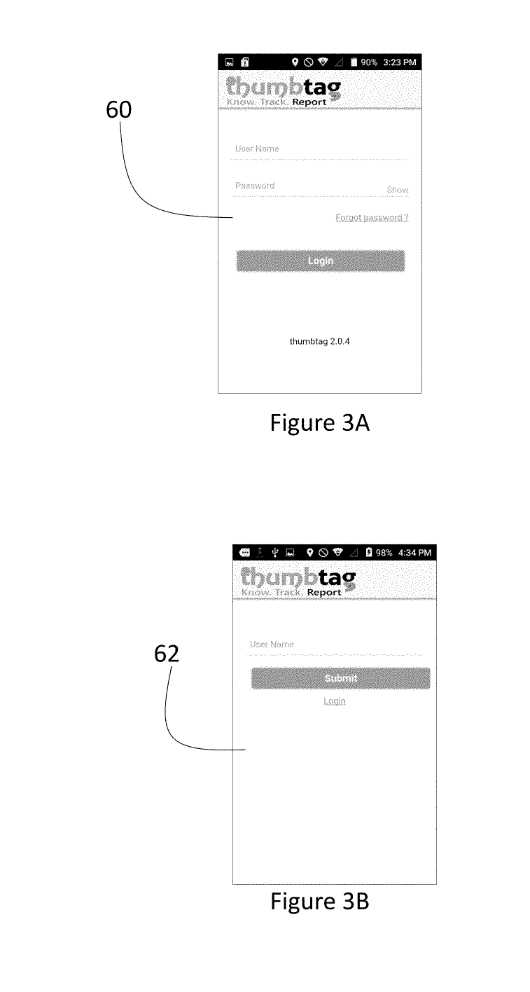

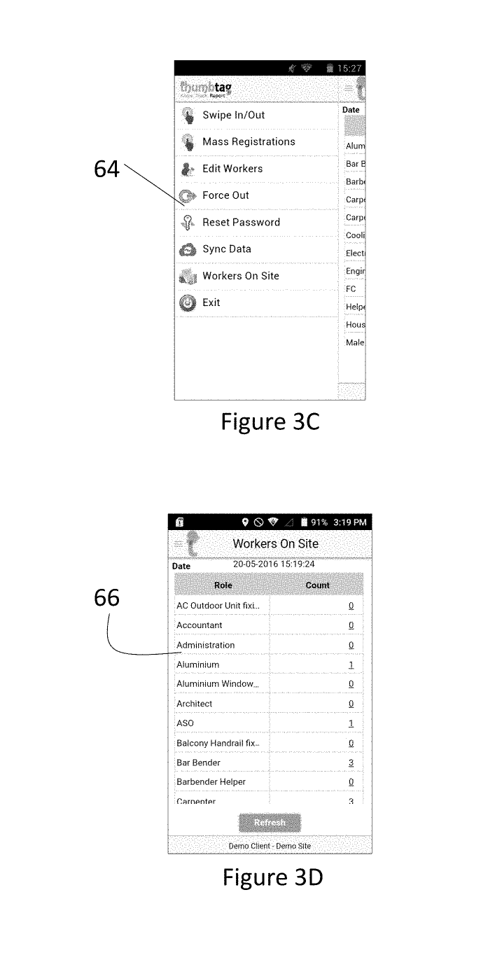

FIGS. 3A to 3D demonstrate the system initialization tasks 42, pursuant to one embodiment. Specifically, FIGS. 3A and 3B depict the management login screen 60 and password management screen 62. FIG. 3C shows an interface 64 of the available management tasks on the end user device 30. FIG. 3D depicts an active list 66 of workers on the job site by the department, per one embodiment of the invention. Other lists of workers available include by worker name, age, swipe in time, schedule, and absenteeism record.

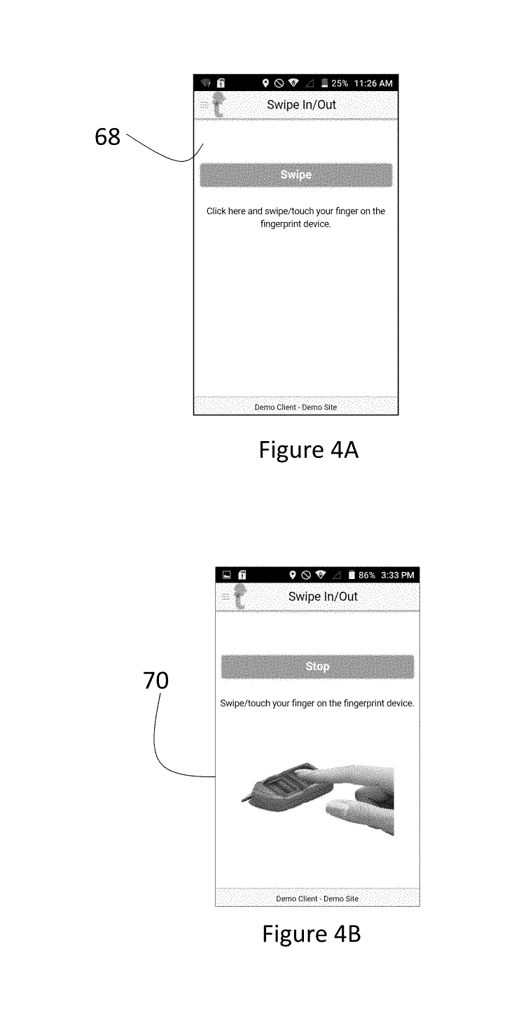

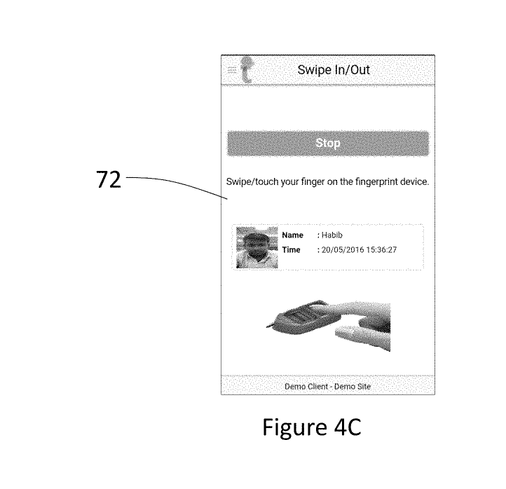

FIGS. 4A to 4C depict the system use tasks 52 within the application. In FIG. 4A, the end user is prompted 68 to swipe (either in or out). In FIG. 4B, the user is presented with the request 70 to confirm biometric information. During the confirmation step screen, shown in FIG. 4C, the end user's photo is included. This ensures that the same person as was originally enrolled is interacting with the biometric reader.

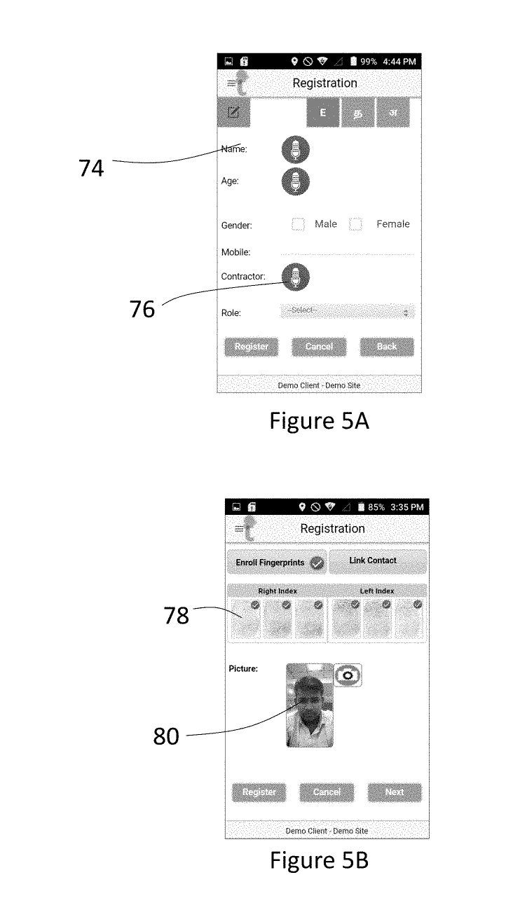

FIG. 5A depicts the registration process for a user by using an interface screen 74. In the depicted embodiment, information about the user, such as name, age and other information is provided using voice commands 76 rather than typed in. Other than the demographic information shown in FIG. 5A, the registration process also includes enrolling of the fingerprints 78 and capturing a photo of the end user 80. In this way, the system is able to match the end user with both fingerprint biometric data as well as facial features in the photo. In this manner, the system is highly resistant to tampering or false swiping activity.

### Server Interface

As shown in FIG. 1, the system includes a management interface 26 running on a separate device 24 than the end user device 30.

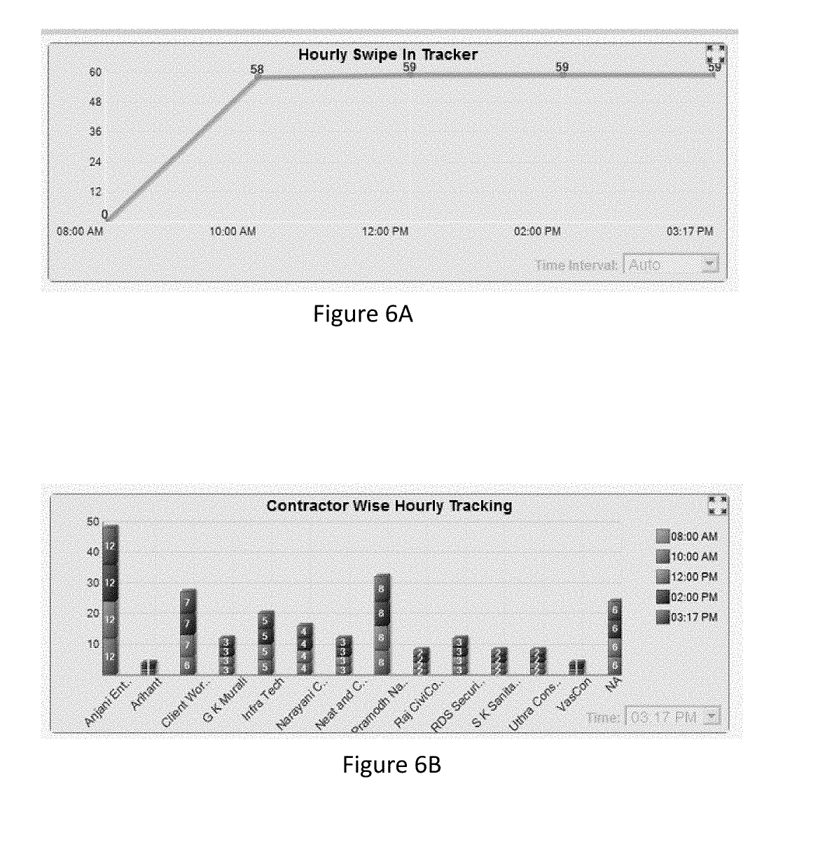

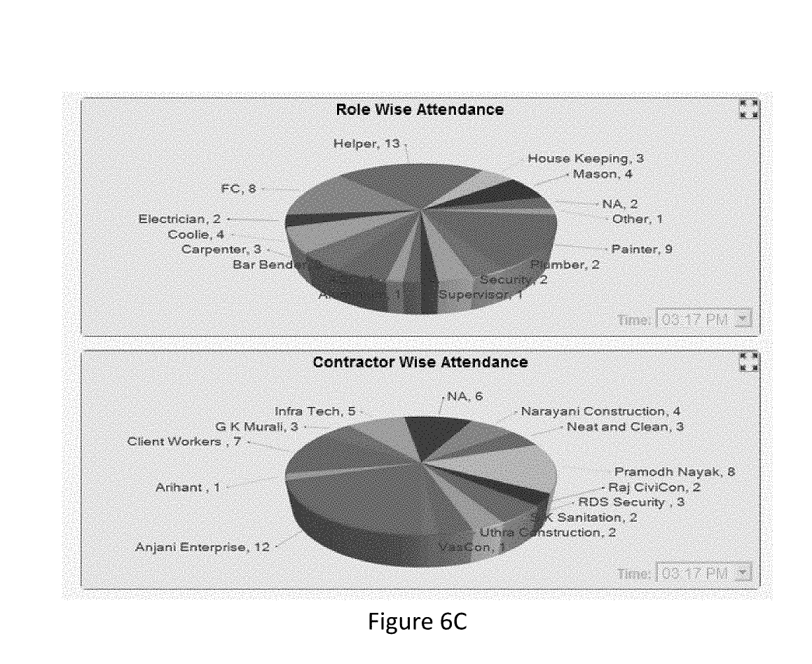

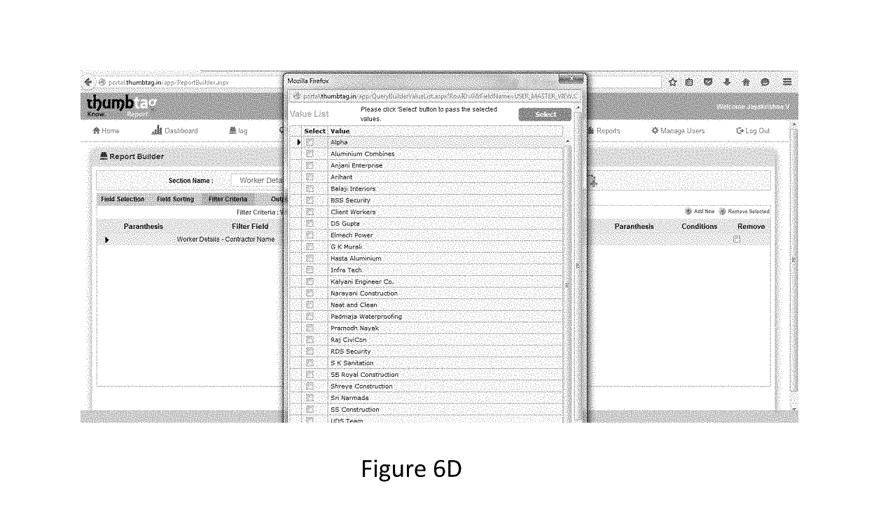

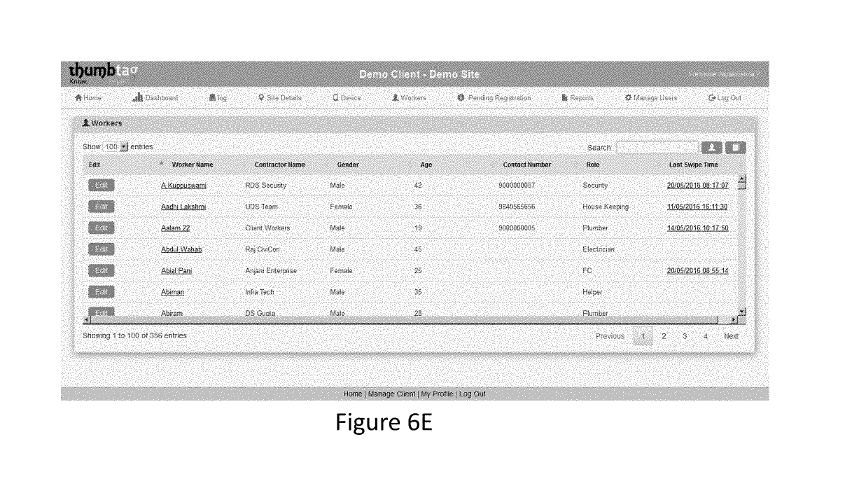

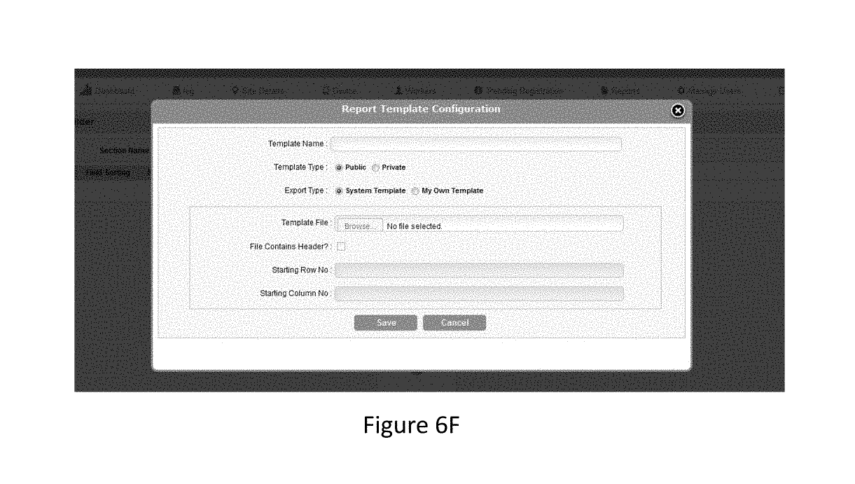

As depicted in sample reports from FIGS. 6A-C, the management interface 26 allows for analysis of the hourly swipe-ins (FIG. 6A), in one embodiment. As shown in FIG. 6B, the interface 26 also allows for contractor tracking. Finally, the interface shows the attendance by role or contractor (FIG. 6C). In one embodiment, the system includes a report builder that enables the data from the console application to be exported to various other applications like payroll, job costing, and others end user reports.

The management interface 26 allows for highly granular review of entry in the system and for accessing of custom reports, such as by accessing the custom report setup screen shown in FIG. 6D.

As the management interface 26 operates within a web browser, in one embodiment, it can provide scalable detailed views, such as the detailed view of employees in FIG. 6E.

One of the features of the server interface is a report builder, as shown in FIG. 6F. The report builder allows an authorized user to specify which system data will be presented to an end user.

In one embodiment, the server interface 26 is optimized for use with a keyboard and a mouse on a high-resolution display. In another embodiment, the server interface operates on a smaller portable device and is optimized for use with a touch screen interface.

### Stand

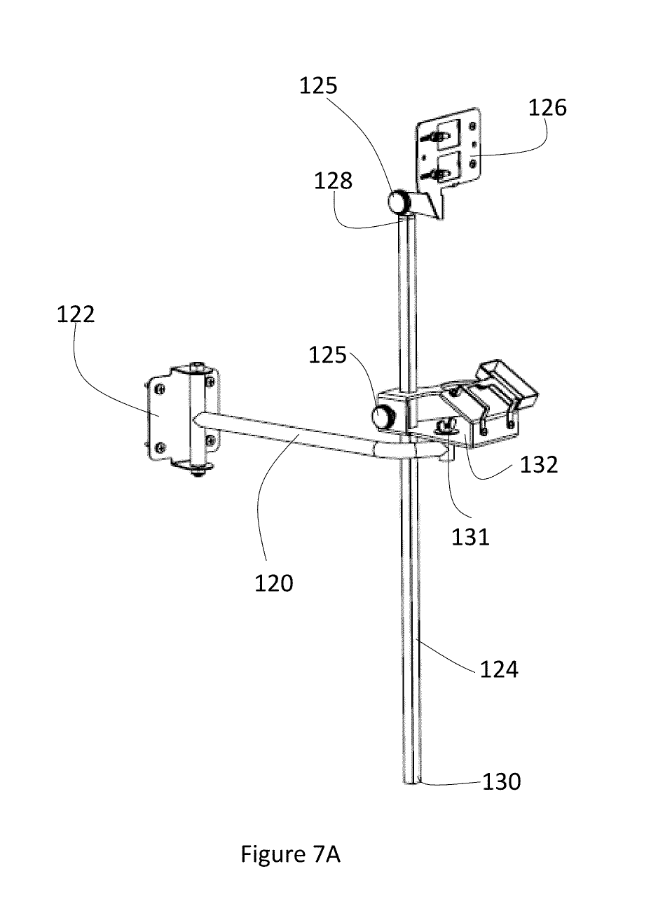

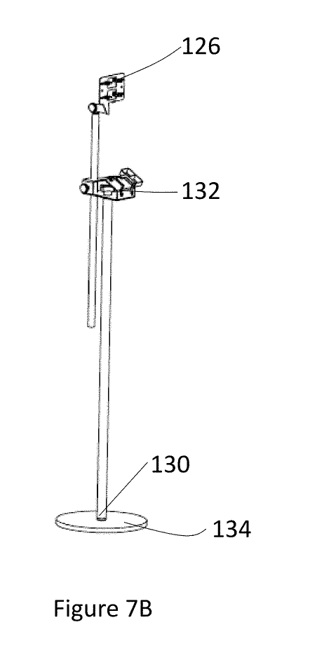

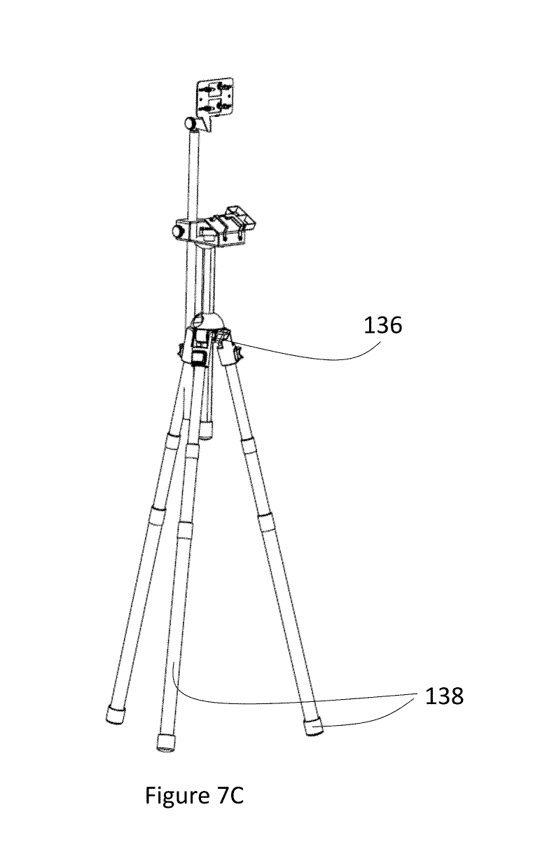

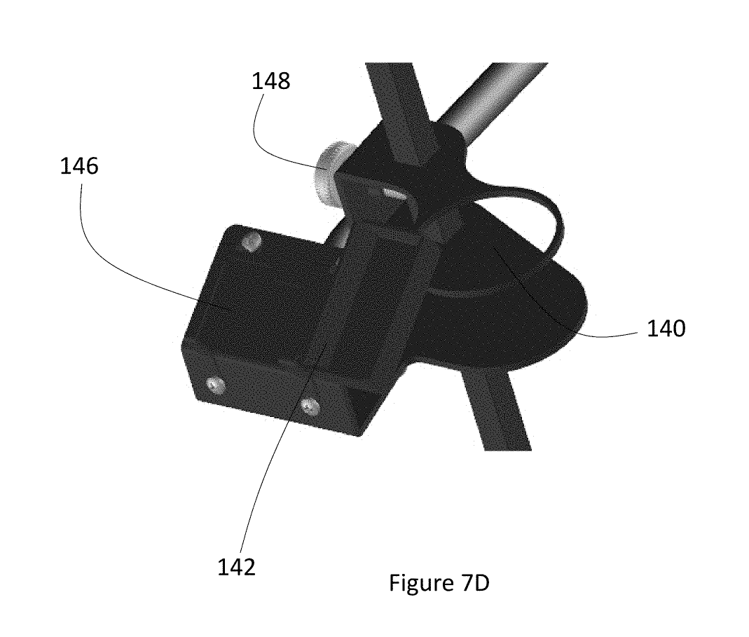

As shown in FIGS. 7A-F the end-user facing components are disposed on a stand 120. The stand 120, as shown in FIG. 7A can be mounted to a wall at a mounting point 122. The mount 120 includes a vertical crossbar 124 which provides a phone mounting point 126 on one end 128 and impacts the ground surface on the opposite end 130. In between the phone mounting point 126 and the ground surface, a biometric station 132 is attached to the vertical cross bar 124.

An alternative embodiment is shown in FIG. 7B. In this embodiment, the ground end 130 ends in a weighted base 134. Otherwise, it also includes a phone mounting point 126 and biometric station 132.

A further alternative embodiment is shown in FIG. 7C. Pursuant to this embodiment, the weighted base 134 of FIG. 7B is replaced with an adjustable tripod 136 having three individually adjusted legs 138.

Each embodiment can be employed in different environments, for example when dealing with uneven ground, the tripod embodiment may be deployed. The wall mount in FIG. 7A is optimized for permanent installation, such as in an office environment where the mount 130 is installed outside a manager's suite.

In FIG. 7A, the vertical cross bar 124 comprises a single metal piece, in one embodiment. The remaining components, such as the phone mount 126 or the biometric station 132 are slid onto the vertical cross bar 124 and secured in place through the use of the user-adjustable knobs 125. In one embodiment, the user-adjustable knobs 125 include a one-way mechanism such that once they are tightened in place, they cannot be released without using a specialized tool. In another embodiment, the knobs 125 are user adjustable.

Inasmuch as the various components, such as the biometric station 132, the phone mounting point 126 and the vertical cross bar 124 are assembled without the need for tools, they may be shipped to a job location in a flat package, with minimal work needed to set up the system on site.

Further, to minimize the shipping space requirements, the largest component (such as the vertical bar 124) is provided in multiple telescoping segments, in one embodiment. In another embodiment, not shown, multiple sub-segments are joined together to form the cross bar 124. In one such embodiment, each crossbar has either a threaded screw end or a receiving thread on the inside surface of the segment and so the sub-segments screw in together. In another embodiment, the sub-segments are combined together using deformable clamps, nuts and bolts, and other suitable fasteners.

Each biometric station 132 includes the biometric reader as well as hand washing dispenser, such as shown in the detailed view of FIG. 7D. The hand washing dispenser is installed in opening 140, a sponge is installed in opening 142, while the biometric reader is installed in opening 146. The sponge is permeated with a few drops of hand sanitizer. The workers wet their finger by touching the sponge before putting their finger on the biometric reader.

The position of the biometric station 132 is fixed in place by securing the fastener, such as the wingnut 131 shown in the cutaway view of the biometric station 132 depicted in FIG. 7A. The details of the biometric station 132 are shown in FIG. 7D.

In one embodiment, the phone holder 126 is adjustable so as to accommodate many different sizes of phones or tablets.

In the depicted embodiments, the stand can be adjusted by manipulating one or more knobs which do not require tools before the stand is adjusted. An example knob 148 is depicted in FIG. 7D.

### Finger Cleaning Solution

As was mentioned above, the stand includes a dispenser for hand washing solution as well as a sponge for cleaning fingers. In one embodiment, the following composition of the solution is used:

Denatured alcohol 69.4%

Water

PEG/PPG-17/6 copolymer

Propylene glycol

Acrylates/c10-30 alkyl acrylate crosspolymer

Tetrahydroxypropyl ethylenediamine

The solution and sponge combination provide for several benefits. The end users swipe their finger on the sponge which results in the worker's finger being cleaned. However, it also raises the fingerprint on their finger so that the interaction with the biometric reader is further less time consuming. While the solution contains some alcohol, the composition does not rely exclusively on an alcohol. Such a solution would be volatile, especially if left out in the sun or in a hot environment.

In one embodiment, inasmuch as the hand-cleaning and swipe station are located at a single physical location, the total time spent interacting with the system is limited to 7 seconds per user. The fact that the end users interact with a single station ensures that their time will be spent as efficiently as possible. The use of a single station also ensures that multiple worker queues are not needed and the workers do not waste too much time.

Although exemplary implementations of the invention have been depicted and described in detail herein, it will be apparent to those skilled in the relevant art that various modifications, additions, substitutions, and the like can be made without departing from the spirit of the invention and these are therefore considered to be within the scope of the invention as defined in the following claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting, but are instead exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the terms “comprising” and “wherein.” Moreover, in the following claims, the terms “first,” “second,” and “third,” are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

The embodiment of the invention in which an exclusive property or privilege is claimed is defined as follows:

1. A system for biometric staff management for a physical jobsite comprising:
a centralized reporting server;
a multi-purpose computing device having authentication software and in communication with at least one biometric sensor;
wherein jobsite workers who are users of said software authenticate into the system by using the at least one biometric sensor;
wherein said software provides live status of workers present on the physical jobsite;
wherein said software provides daily swipe in and swipe out information for each worker;
wherein said software transmits status and swipe in information to said centralized reporting server and exchanges other information with the reporting server;
and a stand for said multi-purpose computing device and at least one biometric sensor;
wherein said stand further comprises a hand cleaning solution dispenser;
wherein said stand provides a rigid base for the multi-purpose computing device and at least one biometric sensor;
wherein said centralized reporting server provides information on the workers present on the jobsite, a total cost for workers, and contractor analysis.

2. The system of claim 1 wherein said biometric sensor comprises a fingerprint reader.

3. The system of claim 1 wherein said multi-purpose computing device comprises a portable phone having at least one external network connection wherein said external network connection provides communication to the centralized reporting server.

4. The system of claim 1 wherein multiple employee workstations are deployed at a jobsite in communication with a centralized reporting server.

5. The system of claim 1 wherein said computing device includes local reporting capability and the local reporting capability is used during periods of an absence of a communication channel to the central reporting server.

6. The system of claim 1 wherein said hand cleaning solution is dispensed on a finger washing station wherein end users of the system first clean at least one finger before interacting with the biometric reader.

7. The system of claim 6 wherein said finger washing station comprises a sponge containing a finger cleaning solution.

8. The system of claim 7 wherein said finger cleaning solution comprises less than 70 percent denatured alcohol.

9. The system of claim 1 wherein said stand comprises a vertical cross bar, a biometric station, a phone mounting point and a wall horizontal crossbar wherein said biometric station and phone mounting point are attached to the vertical cross bar and the wall horizontal crossbar is attached to the biometric station on a first end and a vertical wall on the second end.

10. The system of claim 9 wherein said phone mounting point and said biometric station are reversibly attached to said vertical cross bar.

11. The system of claim 10 wherein said reversible attachment comprises a user-adjustable attachment which does not require tools.

12. The system of claim 1 wherein said stand comprises a vertical cross bar, a biometric station, a phone mounting point and a base wherein said biometric station and phone mounting point are attached to the vertical cross bar and the base is attached to the vertical cross bar at an end of the vertical cross bar closest to a ground level.

13. The system of claim 1 wherein said stand comprises a vertical cross bar, a biometric station, a phone mounting point and a tripod assembly wherein said biometric station and phone mounting point are attached to the vertical cross bar and the tripod assembly is attached to the vertical cross bar at an end of the vertical cross bar closest to a ground level.

14. A method for biometric staff management for a physical jobsite comprising:
providing a centralized reporting server;
providing employees a multi-purpose computing device having authentication software and in communication with at least one biometric sensor;
wherein jobsite workers who are users of said software authenticate into the system by using the at least one biometric sensor;
wherein said software provides live status of workers present on the physical jobsite;
wherein said software provides daily swipe in and swipe out information for each worker;
wherein said software transmits status and swipe in information to said centralized reporting server and exchanges other information with the reporting server; and
providing a stand for said multi-purpose computing device and at least one biometric sensor;
wherein said stand further comprises a hand cleaning solution dispenser;
wherein said stand provides a rigid base for the multi-purpose computing device and at least one biometric sensor;
wherein said centralized reporting server provides information on the workers present on the jobsite, a total cost for workers, and contractor analysis.

15. The method of claim 14 further comprising generating a list of all jobsite workers present at the jobsite at a given time.
